(19) United States
(12) Patent Application Publication
(10) Pub. No.: US 2019/0092739 A1
(43) Pub. Date: Mar. 28, 2019

(54) Process for the manufacture of 3-piperazin-1-yl-propylamine derivatives

(71) Applicant: Hoffmann-La Roche Inc., Little Falls, NJ (US)

(72) Inventors: Shaoning WANG, Basel (CH); Stefan HILDBRAND, Gelterkinden (CH)

(73) Assignee: Hoffmann-La Roche Inc., Little Falls, NJ (US)

(21) Appl. No.: 16/108,496

(22) Filed: Aug. 22, 2018

Related U.S. Application Data
(63) Continuation of application No. PCT/EP2017/053770, filed on Feb. 20, 2017.

(30) Foreign Application Priority Data
Feb. 22, 2016 (EP) 16156707.8

(51) Int. Cl. C07D 295/13

(52) U.S. Cl. CPC C07D 295/13

(57) ABSTRACT

The invention relates to the manufacture of a compound of formula (I)

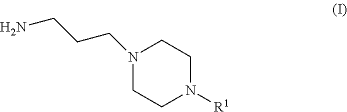

wherein R¹ is defined as in the description and in the claims.

The present invention relates to a process for the manufacture of 3-piperazin-1-yl-propylamine derivatives.

The invention relates in particular to a process for the manufacture of a compound of formula (I)

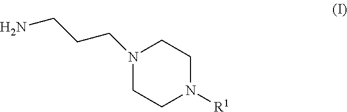

comprising the reaction of a compound of formula (II)

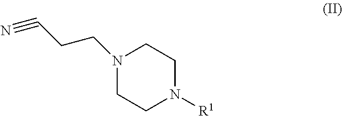

in the presence of hydrogen and a catalyst selected from Raney-Nickel and Raney-Cobalt;

wherein R¹ is alkyl.

Several processes for the manufacture of the compound of formula (I) are known in the art (WO 2014/104272; Ma, Lichao et al., Synthesis 2013, 45(1) 45-52; Ovat, Asli et al. Journal of Medicinal Chemistry 2010, 53(17), 6326-6336; WO 2009/099416; Quia, Jin et al. Journal of Medicinal Chemistry 2008, 51(17), 5264-5270; Hamilton, Chris J. et al., Bioorganic & Medicinal Chemistry 2003, 11(17), 3683-3693; Protiva, M. et al., Collection of Czechoslovak Chemical Communications 1977, 42(12), 3628-42).

However, the known processes have many drawbacks and cannot be used industrially. They have in particular low yield, use expensive metals, need a tedious work up to remove the residual reagents or by products, or employ conditions that cannot be used on a large scale.

The compound of formula (I) is a building block for the synthesis of several biologically active compounds.

There was therefore the need for a convenient and efficient process that gives access to the compound of formula (I) on large, industrial scale.

This problem has been solved by the process according to the invention.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl more particularly methyl, ethyl, propyl, isopropyl, isobutyl, tert.-butyl and isopentyl. A particular example of alkyl is methyl.

The invention relates in particular to:

A process according to the invention wherein the compound of formula (II) is obtained by the reaction of a compound of formula (III)

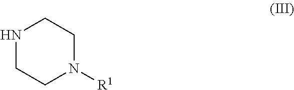

in the presence of acrylonitrile, wherein R¹ is as defined above;

A process according to the invention wherein the compound of formula (II) is not isolated and directly converted to the compound of formula (I);

A process according to the invention wherein the catalyst is Raney-Nickel;

A process according to the invention wherein the reaction of the compound of formula (II) in the presence of hydrogen and a catalyst is done in methanol, tetrahydrofuran, ethanol, i-propanol, toluene, pentan-octane, methyltetrahydrofurane, methyl tert-butyl ether, ethyl acetate, water or dioxane;

A process according to the invention wherein the reaction of the compound of formula (II) in the presence of hydrogen and a catalyst is done in methanol or tetrahydrofuran, in particular methanol;

A process according to the invention wherein the reaction of the compound of formula (II) in the presence of hydrogen and a catalyst is done in the presence of a base;

A process according to the invention wherein the base is ammonia, sodium acetate or an alkali metal hydroxide;

A process according to the invention wherein the alkali metal hydroxide is NaOH;

A process according to the invention wherein the catalyst is Raney-Cobalt and the base is NaOH;

A process according to the invention wherein the catalyst is Raney-Nickel and the base is ammonia;

A process according to the invention wherein the reaction of the compound of formula (II) in the presence of hydrogen and a catalyst is done at a temperature between 15 and 100° C., in particular between 25 and 65° C., more particularly between 30 and 50° C., more particularly at around 40° C.;

A process according to the invention wherein the reaction of the compound of formula (II) in the presence of hydrogen and a catalyst is done at a pressure between 0.1 and 200 bar, more particularly between 5 and 20 bar, more particularly at around 10 bar;

A process according to the invention wherein around 0.01 to around 0.5 equivalent of Raney-Nickel or Raney-Cobalt is used;

A process according to the invention wherein the reaction of a compound of formula (III) in the presence of acrylonitrile is done in methanol, ethanol, n-propanol, isopropanol or butanol;

A process according to the invention wherein the reaction of a compound of formula (III) in the presence of acrylonitrile is done at a temperature between 15 and 66° C.; and

A process according to the invention wherein R¹ is methyl, i.e. wherein the compound of formula (III) is

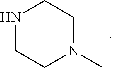

A process according to the invention wherein the reaction of the compound of formula (II) in the presence of hydrogen and a catalyst is done at a temperature of around 40° C. is particularly advantageous.

A process according to the invention wherein the reaction of the compound of formula (II) in the presence of hydrogen and a catalyst is done at a pressure of around 10 bar is particularly advantageous.

A process according to the invention wherein the reaction of the compound of formula (II) in the presence of hydrogen and a catalyst is done at a temperature of around 40° C. and at a pressure of around 10 bar is particularly advantageous.

The reaction of the compound of formula (II) in the presence of hydrogen and a catalyst is advantageously done in around 2 to 6 hrs.

A process according to the invention wherein the reaction of the compound of formula (II) in the presence of hydrogen and a catalyst is done in around 4 to 5 hrs is particularly advantageous.

A process according to the invention wherein the reaction of the compound of formula (II) in the presence of hydrogen and a catalyst is done at a temperature of around 40° C. and at a pressure of around 10 bar in around 4 to 5 hrs is particularly advantageous.

A process according to the invention wherein the reaction of the compound of formula (II) in the presence of hydrogen and a catalyst is done at a temperature of around 40° C., at a pressure of around 10 bar, in around 4 to 5 hrs in methanol is particularly advantageous.

A process according to the invention wherein the reaction of the compound of formula (II) in the presence of hydrogen and Raney-Nickel is done at a temperature of around 40° C., at a pressure of around 10 bar, in around 4 to 5 hrs in methanol and ammonia is particularly advantageous.

A process according to the invention wherein the reaction of a compound of formula (III) in the presence of acrylonitrile is done in methanol is particularly advantageous.

A process according to the invention wherein the reaction of a compound of formula (III) in the presence of acrylonitrile is done during around 2 to 6 hrs, more particularly during around 3 to 5 hrs, more particularly around 3 hrs is advantageous.

A process according to the invention wherein the reaction of a compound of formula (III) in the presence of acrylonitrile is done at around 25° C. is particularly advantageous.

A process according to the invention wherein the reaction of a compound of formula (III) in the presence of acrylonitrile is done in methanol during around 3 hrs at around 25° C. is particularly advantageous.

Very high yield and purity of the compound of formula (I) are particularly obtained when the compound of formula (II) is not isolated or purified and directly converted to the compound of formula (I).

When the compound of formula (II) is not isolated or purified, the reaction mixture containing the compound of formula (II) can be advantageously concentrated, for example by distillation, before the compound of formula (II) is converted to the compound of formula (I).

The reaction mixture containing the compound of formula (II) can be advantageously concentrated until no more solvents are distilled off before the compound of formula (II) is converted to the compound of formula (I).

When the catalyst is Raney-Cobalt, it is preferred that no base is used.

When the catalyst is Raney-Cobalt and if a base is used, the base is advantageously NaOH.

When the catalyst is Raney-Nickel, the addition of a base, in particular ammonia, is advantageous.

Alternatively to Raney-Nickel and Raney-Cobalt, spongy nickel/cobalt or skeletal nickel/cobalt can of course be used. So the invention also relates to a process as defined above wherein the catalyst is a skeletal nickel catalyst, skeletal cobalt catalyst, sponge-nickel catalyst or sponge-cobalt catalyst.

The concentration of the compound of formula (II) in the hydrogenation reaction is advantageously between 10% and 20% (w/solvent volume), more particularly around 10% (w/solvent volume).

The base is advantageously used at 0.1 to 10 equivalents.

The invention will now be illustrated by the following examples which have no limiting character.

### EXAMPLES

Abbreviations: MeOH: methanol; THF: tetrahydrofuran; EtOAc: ethyl acetate; GC: gas chromatography; hrs: hours; eq.: equivalent; Ra—Ni: Raney-Nickel; Ra—Co: Raney-Cobalt; Pd/C: palladium on carbon; Pt/C: platinum on carbon; Rh/Alox: rhodium on aluminum oxide; P: pressure; T: temperature; t: time.

### Example 1

### Synthesis of 3-(4-methylpiperazin-1-yl)propan-1-amine

The following reaction was carried out under various conditions.

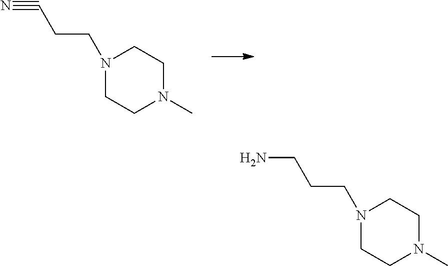

The different reaction conditions have been tested on a short time frame, without always letting the reaction going to completion. This was made simply to check efficiently whether or not the reactions are working. Therefore a low conversion rate but high relative yield is indicative of a positive result.

### Example 1.1

200 mg of 3-(4-methylpiperazin-1-yl)propanenitrile (1.3 mmol) and 2 ml MeOH (Sigma-Aldrich) was transferred together with 20 mg Raney-Cobalt (0.147 mmol, Johnson Matthey A-8B46 Sponge Cobalt) into a 35 mL stainless steel autoclave, which was sealed and 3 times pressurized with 10 bar H₂ and releasing to normal pressure, thereafter charged with 10 bar of hydrogen. The autoclave was heated under program control to 40° C. and shaked for 2 hrs. After this time the autoclave was cooled to room temperature, the pressure released and the reaction mixture filtered. The filtrate was analyzed with GC, showed a conversion of 88% and a selected yield of 85% of 3-(4-methylpiperazin-1-yl)propan-1-amine.

### Example 1.2

This example was run in an analogous manner as example 1.1 but using 64 mg of 3-(4-methylpiperazin-1-yl)propanenitrile (0.41 mmol) and 2 ml 7 N NH₃ in MeOH (Sigma-Aldrich) and 15 mg Raney-Nickel (0.119 mmol, EVONIK B113Z) at 23° C. under 10 bar for 5 hrs. GC analysis showed a conversion of 87% and a yield of 85% of 3-(4-methylpiperazin-1-yl)propan-1-amine.

### Example 1.3

This example was run in an analogous manner as example 1.2 but using 100 g of 3-(4-methylpiperazin-1-yl)propanenitrile (652 mmol) and 1 L 7 N NH₃ in MeOH (Sigma-Aldrich) and 10 g Raney-Nickel (79.6 mmol, EVONIK B113Z) in a 1.5 L autoclave at 40° C. under 10 bar for 5 hrs stirring. GC analysis showed a conversion of 100% and 98.9% yield of 3-(4-methylpiperazin-1-yl)propan-1-amine.

### Example 1.4

This example was run in an analogous manner as example 1.2 but using 200 mg of 3-(4-methylpiperazin-1-yl)propanenitrile (1.3 mmol) and 2 ml MeOH (Sigma-Aldrich) and 20 mg Raney-Nickel (0.159 mmol, EVONIK B113Z) at 40° C. under 10 bar for 1 hrs. GC analysis showed a conversion of 97.6% and 62.8% yield of 3-(4-methylpiperazin-1-yl)propan-1-amine.

### Example 1.5

This example was run in an analogous manner as example 1.2 but using 400 mg of 3-(4-methylpiperazin-1-yl)propanenitrile (2.6 mmol) and 2 ml with NH₃ saturated THF (Self made) and 20 mg Raney-Nickel (0.159 mmol, EVONIK B113Z) at 40° C. under 10 bar for 2 hrs. GC analysis showed a conversion of 31.7% and 28.6% yield of 3-(4-methylpiperazin-1-yl)propan-1-amine.

### Example 1.6

This example was run in an analogous manner as example 1.2 but using 200 mg of 3-(4-methylpiperazin-1-yl)propanenitrile (1.3 mmol) and 2 ml THF and 20 mg Raney-Cobalt (0.147 mmol, Johnson Matthey A-8B46 Sponge Cobalt) at 23° C. under 10 bar for 2 hrs. GC analysis showed a conversion of 13.1% and 12.8% yield of 3-(4-methylpiperazin-1-yl)propan-1-amine.

### Comparative Example 1.7

This example was run in an analogous manner as example 1.1 but using 200 mg of 3-(4-methylpiperazin-1-yl)propanenitrile (1.3 mmol) and 2 ml MeOH and 20 mg 10% Pd/C (0.019 mmol, EVONIK E 101 N/D) at 40° C. under 10 bar for 2 hrs. GC analysis showed a conversion of 100% and 1.8% yield of 3-(4-methylpiperazin-1-yl)propan-1-amine.

### Comparative Example 1.8

This example was run in an analogous manner as example 1.7 but using 200 mg of 3-(4-methylpiperazin-1-yl)propanenitrile (1.3 mmol) and 2 ml MeOH and 38.6 mg 5% Rh/Alox (0.019 mmol, EVONIK G 213 XKR/D) at 40° C. under 10 bar for 2 hrs. GC analysis showed a conversion of 100% and 13.6% yield of 3-(4-methylpiperazin-1-yl)propan-1-amine.

### Comparative Example 1.9

This example was run in an analogous manner as example 1.7 but using 200 mg of 3-(4-methylpiperazin-1-yl)propanenitrile (1.3 mmol) and 2 ml MeOH and 73.3 mg 5% Pt/C (0.019 mmol, EVONIK F 101 R/D) at 40° C. under 10 bar for 2 hrs. GC analysis showed a conversion of 19.2% and 0.54% yield of 3-(4-methylpiperazin-1-yl)propan-1-amine.

### Example 1.10

This example was run in an analogous manner as example 1.1 but using 100 g of 3-(4-methylpiperazin-1-yl)propanenitrile (652 mmol) and 1 L MeOH and 10 mg Raney-Cobalt (73.6 mmol, Johnson Matthey A-8B46 Sponge Cobalt) in a 1.5 L autoclave at 40° C. under 10 bar for 4 hrs stirring. GC analysis showed a conversion of 100% and 93.7% yield of 3-(4-methylpiperazin-1-yl)propan-1-amine.

The above results, as well as other experiments are summarized in Table 1 below.

| | | Starting | | | | T | P | t | Conversion | Yield |
|---|---|---|---|---|---|---|---|---|---|---|
| Experiment | Example | material | | Solvent | Catalyst | [° C.] | [bar] | [hrs] | [%] | [%] |
| 1 | 1.2 | 64 | mg | 2 ml 7N | 15 mg, | 23 | 10 | 5 | 87 | 85 |
| | | | | NH3 in | 0.29 eq. | | | | | |
| | | | | MeOH | Ra—Ni | | | | | |
| 2 | | 88 | mg | 2 ml 7N | 22 mg, | 40 | 10 | 5 | 100 | 97.4 |
| | | | | NH3 in | 0.305 eq. | | | | | |
| | | | | MeOH | Ra—Ni | | | | | |
| 3 | | 100 | g | 1 L 7N | 25 g, | 40 | 10 | 2 | 100 | 99 |
| | | | | NH3 in | 0.305 eq. | | | | | |
| | | | | MeOH | Ra—Ni | | | | | |
| 4 | | 100 | mg | 2 ml 7N | 25 mg, | 40 | 10 | 0.5 | 93.1 | 91.4 |
| | | | | NH3 in | 0.305 eq. | | | | | |
| | | | | MeOH | Ra—Ni | | | | | |
| 5 | 1.3 | 100 | g | 1 L 7N | 10 g, | 40 | 10 | 5 | 100 | 98.9 |
| | | | | NH3 in | 0.122 eq. | | | | | |
| | | | | MeOH | Ra—Ni | | | | | |
| 6 | 1.4 | 200 | mg | 2 ml | 20 mg, | 40 | 10 | 1 | 97.6 | 62.8 |
| | | | | MeOH | 0.122 eq. | | | | | |
| | | | | | Ra—Ni | | | | | |
| 7 | 1.5 | 400 | mg | 2 ml | 20 mg, | 40 | 10 | 2 | 31.7 | 28.6 |
| | | | | saturated | 0.061 eq. | | | | | |
| | | | | NH3 in | Ra—Ni, | | | | | |
| | | | | THF | No. 313 | | | | | |
| 8 | | 400 | mg | 2 ml | 20 mg, | 23 | 5 | 2 | 43.9 | 34.4 |
| | | | | MeOH | 0.061 eq. | | | | | |
| | | | | | Ra—Ni | | | | | |
| 9 | | 200 | mg | 2 ml | 20 mg, | 23 | 5 | 1 | 21.7 | 18.6 |
| | | | | saturated | 0.122 eq. | | | | | |
| | | | | NH3 in | Ra—Ni | | | | | |
| | | | | THF | | | | | | |
| 10 | | 400 | mg | 2 ml THF | 20 mg, | 40 | 5 | 1 | 2.4 | 1.8 |
| | | | | | 0.060 eq. | | | | | |
| | | | | | Ra—Co | | | | | |
| 11 | 1.6 | 200 | mg | 2 ml THF | 20 mg, | 23 | 10 | 2 | 13.1 | 12.8 |
| | | | | | 0.113 eq. | | | | | |
| | | | | | Ra—Co | | | | | |
| 12 | 1.7 | 200 | mg | 2 ml | 20 mg | 40 | 10 | 2 | 100 | 1.8 |
| | | | | MeOH | 0.0144 eq. | | | | | |
| | | | | | 10% Pd/C | | | | | |
| 13 | 1.8 | 200 | mg | 2 ml | 38.6 mg, | 40 | 10 | 2 | 100 | 13.6 |
| | | | | MeOH | 0.0144 eq. | | | | | |
| | | | | | 5% Rh/Alox | | | | | |
| 14 | 1.9 | 200 | mg | 2 ml | 73.3 mg, | 40 | 10 | 2 | 28.8 | 0.67 |
| | | | | MeOH | 0.0144 eq. | | | | | |
| | | | | | 5% Pt/C | | | | | |
| 15 | 1.1 | 200 | mg | 2 ml | 20 mg, | 40 | 10 | 2 | 88 | 85 |
| | | | | MeOH | 0.113 eq. | | | | | |
| | | | | | Ra—Co | | | | | |
| 16 | 1.1 | 100 | g | 1 1 MeOH | 10 g, | 40 | 10 | 4 | 100 | 93.7 |
| | | | | | 0.113 eq. | | | | | |
| | | | | | Ra—Co | | | | | |
| 17 | | 200 | mg | 1 eq | 20 mg, | 40 | 10 | 2 | 100 | 96.6 |
| | | | | NaOH in | 0.113 eq. | | | | | |
| | | | | 2 ml | Ra—Co | | | | | |
| | | | | MeOH | | | | | | |
| 18 | | 200 | mg | 12.2 mg, | 20 mg, | 40 | 10 | 2 | 13.4 | 13 |
| | | | | 0.113 eq | 0.113 eq. | | | | | |
| | | | | NaOAc in | Ra—Co | | | | | |
| | | | | 2 ml | | | | | | |
| | | | | EtOAc | | | | | | |

### Example 2

### Telescoped Process for the Preparation of 3-(4-methylpiperazin-1-yl)propan-1-amine

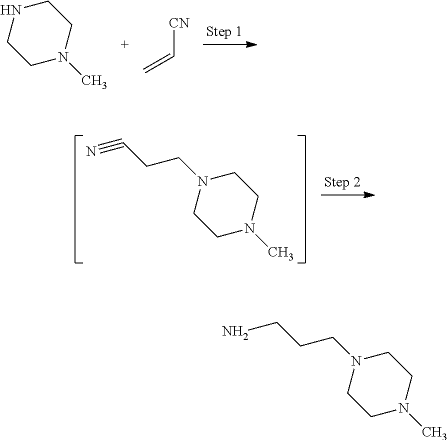

### Step 1

Acrylonitrile (35.56 g, 1.05 eq.) was added within one hour at 25° C. to a solution off N-methyl-piperazine (63.90 g, 1.00 eq.) in MeOH (240 mL) and the resulting mixture was stirred for 3 hours at 25° C. The mixture was concentrated at 35° C./250 mbar unless no more solvent was distilled off and the residue (100 g) was directly used in the next step.

### Step 2

The above residue (100 g) was dissolved in methanol containing ammonia (7N, 1000 mL in total) and hydrogenated in the presence of 10 g of Raney-Nickel (at 40° C. and 10 bar for 5 hours). The catalyst was filtered off and the filtrate was concentrated at 32-37° C./400 mbar to dryness. The residue (104.9 g) was purified by fractional distillation to afford 88.30 g (88% yield over two steps) of 3-(4-methylpiperazin-1-yl)propan-1-amine with a purity of 99.97% (measured by GC).

1. A process for the manufacture of a compound of formula (I) 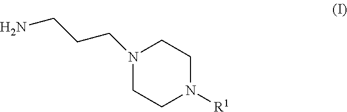
comprising the reaction of a compound of formula (II) 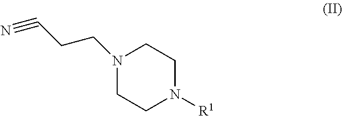
in the presence of hydrogen and a catalyst selected from Raney-Nickel and Raney-Cobalt;
wherein R¹ is alkyl;
at a pressure between 5 and 20 bar; and
wherein around 0.01 to around 0.5 equivalent of Raney-Nickel or Raney-Cobalt is used.

2. A process according to claim 1, wherein the compound of formula (II) is obtained by the reaction of a compound of formula (III) 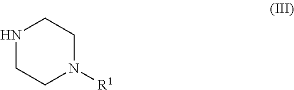
in the presence of acrylonitrile, wherein R¹ is as defined in claim 1.

3. A process according to claim 2, wherein the compound of formula (II) is not isolated and directly converted to the compound of formula (I).

4. A process according to claim 1, wherein the catalyst is Raney-Nickel.

5. A process according to claim 1, wherein the reaction of the compound of formula (II) in the presence of hydrogen and a catalyst is done in methanol, tetrahydrofurane, ethanol, i-propanol, toluene, pentan-octane, methyltetrahydrofurane, methyl tert-butyl ether, ethyl acetate, water or dioxane.

6. A process according to claim 1, wherein the reaction of the compound of formula (II) in the presence of hydrogen and a catalyst is done in methanol or tetrahydrofurane.

7. A process according to claim 1, wherein the reaction of the compound of formula (II) in the presence of hydrogen and a catalyst is done in the presence of a base.

8. A process according to claim 1, wherein the reaction of the compound of formula (II) in the presence of hydrogen and a catalyst is done at a temperature between 15 and 100° C.

9. A process according to claim 1, wherein the reaction of the compound of formula (II) in the presence of hydrogen and a catalyst is done at a pressure of around 10 bar.

10. A process according to claim 2, wherein the reaction of a compound of formula (III) in the presence of acrylonitrile is done in methanol, ethanol, n-propanol, isopropanol or butanol.

11. A process according to claim 2, wherein the reaction of a compound of formula (III) in the presence of acrylonitrile is done at a temperature between 15 and 66° C.

12. A process according to claim 1, wherein R¹ is methyl.

13. (canceled)
